(19) United States
(12) Patent Application Publication
(10) Pub. No.: US 2006/0200125 A1
(43) Pub. Date: Sep. 7, 2006

(54) Equipment for repairing biological tissue, such as a tendon or a ligament and, in particular, the calcanean tendon

(76) Inventors: Patrick Delponte, GUIPAVAS (FR); Jean-Jacques Martin, Bourg En Bresse (FR)

Correspondence Address: YOUNG & THOMPSON, 745 SOUTH 23RD STREET, 2ND FLOOR, ARLINGTON, VA, 22202, US

(21) Appl. No.: 10/534,379

(22) PCT Filed: Nov. 5, 2003
(86) PCT No.: PCT/FR03/03297

§ 371 Date: Jan. 20, 2006

(30) Foreign Application Priority Data
Nov. 7, 2002 (FR) 02/13955

(51) Int. Cl. A61F 5/04

(52) U.S. Cl. 606/53

(57) ABSTRACT

The invention relates to equipment for repairing biological tissue, such as a tendon or a ligament and, in particular, the calcanean tendon. The inventive equipment comprises a link (3) and elements (5) which are used to stop the link (3) in relation to the fragments of tissue to be repaired. According to the invention: (i) one of the aforementioned stop elements comprises a bone screw (2) which can be implanted in a bone surrounding the tissue to be repaired, for example in the calcaneum in order to repair the calcanean tendon; (ii) the above-mentioned link (3) forms a tubular part (6) in which one part of the screw (2) can be tightly inserted along with at least two cords (4) which can be inserted through the fragments of the biological tissue to be repaired, for example the two fragments of a broken calcanean tendon; and (iii) other stop elements comprise at least two stop buttons (5).

The present invention relates to equipment for repairing biological tissue, such as a tendon or a ligament, and in particular a calcanean tendon.

It is known to repair a broken tendon using equipment comprising:

a link connected, at one end, to a needle and comprising a harpoon at the other end thereof,

a bearing washer, and

a deformable sleeve which may be crimped on the link so as to lock the bearing washer in a position determined relative to said link.

The needle enables to engage the link through tendon fragments, in the longitudinal direction thereof, until the harpoon engages into the proximal fragment, then the washer and the sleeve are engaged on the needle and on the link. After proper tensioning of the link, the washer is pressed against the patient's skin, at the malleolar face, and the sleeve is crimped on the link to lock said link with respect to the washer. The portion of the link protruding beyond the sleeve is then cut off.

In practice, two pieces of equipment are placed in parallel, for perfect setting of both tendon fragments relative to one another. After the four to five weeks' period necessary to the healing of both tendon fragments, the link is cut off beneath the washer and is slid off.

This piece of equipment, and the technique which it enables to implement, prove satisfactory in practice. It appears however that the sequels of the intervention remain restricting for the patient and for the practician.

Indeed, when repairing the calcanean tendon, the patient may not submit his foot to any weight immediately and physiotherapy may only start after a minimum period of time, which leads to more or less perilous walking of the patient during said period of time. The patient may not resume his activities, in particular a training schedule in case of an athlete, and must postpone them accordingly. There is still a risk of iterative rupture, even if the percentage of rupture is recognised as acceptable.

The equipment must be withdrawn, moreover, once the fragments have healed, which implies a new intervention. Said equipment may not be left in place more than four to five weeks as mentioned above, and the relatively precocious ablation thereof is not particularly reassuring for the patient, nor for the practician. Consequently, the patient is strongly advised to resume his activities very gradually.

The present invention intends to remedy these significant practical shortcomings.

The equipment affected includes, in a manner known in itself,

a link whereof portions may be inserted through fragments of the biological tissue to be repaired, and

elements for stopping the link relative to such fragments of tissue.

According to the invention,

one of the stop elements is formed of a bone screw which may be implanted in a bone surrounding the tissue to be repaired, in particular in the calcaneum in order to repair the calcanean tendon;

the link forms a tubular part in which one part of the screw can be tightly inserted along with at least two cords which can be inserted through the fragments of the biological tissue to be repaired, in particular the two fragments of a broken calcanean tendon, and

other stop elements comprise at least two stop buttons.

The screw is thus engaged into said tubular part of the link, than is screwed into the bone so as to anchor one end of said link relative to said bone.

Each cord is then inserted through tissue fragments to be joined then, after proper tensioning, is interconnected with a stop button so as to maintain the fragments of tissue in a coaptation state.

Such equipment enables to improve significantly the sequels of the intervention and the patient's comfort inasmuch as it enables more precocious physiotherapy whereby, in the case of a calcanean tendon, the patient may lay his foot on the ground straightaway, which provides for less perilous walking of the patient.

Preferably, the screw and/or the link and/or the stop buttons are made of a bioresorbable material.

The equipment then remains in place in the organism until the resorption thereof, and does involve any new intervention for the ablation thereof.

The piece of equipment remains efficient for a duration of approximately three months, longer than the duration of implantation of a piece of equipment according to prior art, which enables the patient to resume his activities, in particular a training schedule in case of an athlete, earlier and more reliably for the patient, and more reassuring for the surgeon.

The screw is preferably a so-called “interference” screw, i.e. deprived of a head and controlled via a prismatic cavity emerging into the proximal end thereof.

The tubular part of the link may be in particular realised by knitting threads in the form of a sock, and the cords may be realised by weaving said very threads.

Preferably, the link includes four cords to enable perfect stabilisation of the fragments of biological tissue to be repaired, in particular perfect locking of a fragment of calcanean tendon relative to the other.

Each cord might be connected permanently to a needle at the free end thereof, in particular by crimping the proximal end of the needle on the free end of the cord. However, each cord is preferably connected to a needle by insertion thereof through the eye of said needle and exhibits a rigidified free end to easier insertion thereof. Said rigidification may be provided in particular by assembling threads forming the cord at this free end.

Each stop button may, for its own part, simply comprise one or two through-holes, for the engagement thereof on a cord and the immobilisation thereof relative to the latter by ligature.

For purely illustrative purposes, the elements forming the equipment according to the invention may exhibit the following sizes and features:

screw: 7 mm in diameter, 24 mm in length, conical in shape at the distal end thereof;

link: tubular part, 8 mm in length and with a diameter suited to the diameter of the screw; cords: braids of threads of bioresorbable material, 1.1 mm in diameter in total, and at least 30 cm in length;

buttons: each of them is 0.5 mm thick, with a 5 mm diameter, and comprises two holes for the ligature thereof to a cord or a pair of cords.

The invention will be better understood, and other features and characteristics thereof will appear, with reference to the appended schematic drawing, which represents, for non-limiting exemplification purposes, a preferred embodiment of the equipment related.

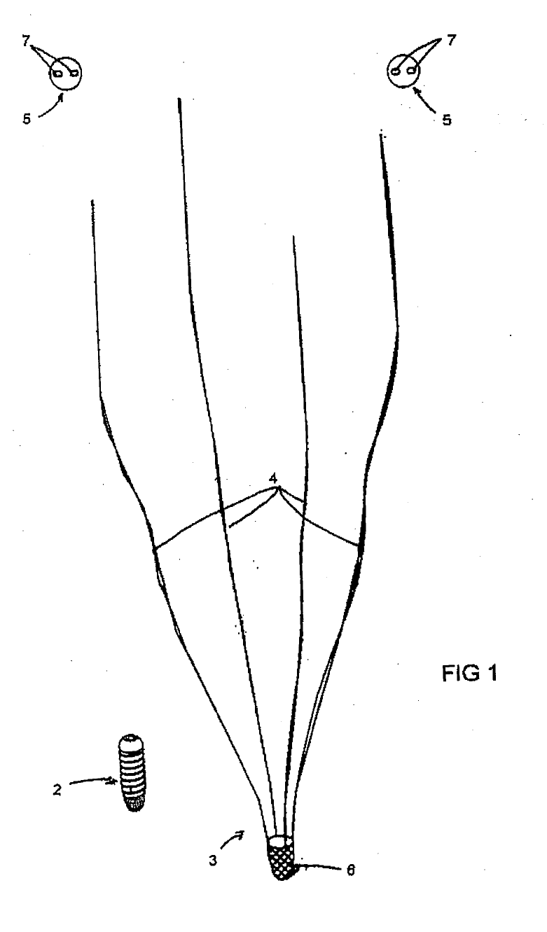

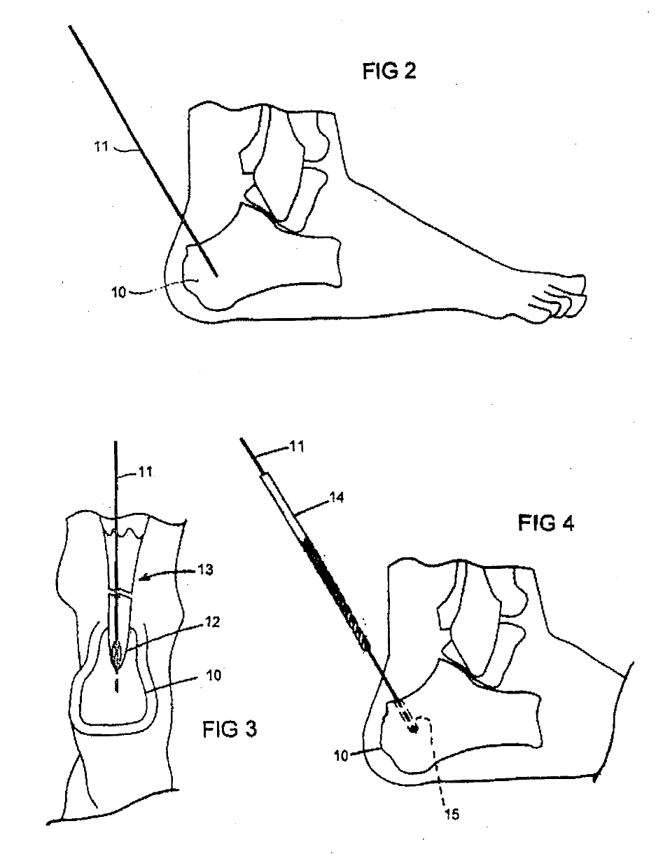

FIG. 1 is a perspective view of the different elements forming said piece of equipment, and FIGS. 2 to 9 are views of different successive stages of the repairing process, using said equipment, of a broken calcanean tendon.

FIG. 1 represents different elements forming a piece of equipment for repairing biological tissue, such as a tendon or a ligament, and in particular a calcanean tendon.

Such equipment comprise a bone screw 2, a link 3 whereof portions 4 may be inserted via a needle through fragments of the biological tissue to be repaired, and two stop buttons 5.

The screw 2 is preferably a so-called “interference” screw, i.e. deprived of a head. It is drilled through along its axis for engaging on a guiding spindle, comprises a prismatic cavity enabling the rotation thereof, and exhibits a conical shape at the distal end thereof; for easier insertion.

The screw 2 is designed for implantation in a bone surrounding the tissue to be repaired, in particular in the calcaneum for repairing the calcanean tendon, and is made of a bioresorbable material. For repairing the calcanean tendon, it is 24 mm in length and 7 mm in diameter.

The link 3 forms a tubular part 6 which is slightly conical, as well as four cords 4 forming said portions which may be inserted through fragments of the tissue to be repaired.

The tubular part 6 is capable of receiving with a tight fit the proximal portion of the screw 2 therein. For repairing the calcanean tendon, it is 8 mm in length. It is realised by knitting threads of bioresorbable material in the form of a sock, and the cords 4 are realised by weaving said very threads.

These cords 4, are, when repairing the calcanean tendon, 1.1 mm in diameter in whole and 35 cm in length.

The free end of each cord 4 is designed for insertion into the eye of a needle (not represented) enabling the introduction of the cord 4 into the fragments of the tissue to be repaired, and exhibits a rigidified free end zone for easier insertion. Said rigidification is provided by assembling threads forming the cord 4 at this free end.

Each stop button 5 is formed of a washer of bio resorbable material and comprises two through-holes 7, for the engagement thereof on a pair of cords 4 and the immobilisation thereof relative to the latter by ligature. For repairing the calcanean tendon, each button 5 is 0.5 mm in thickness and 5 mm in diameter.

In practice, as shown on FIGS. 2 to 4, a self-drilling spindle 11 is placed in the calcaneum 10, beneath the lower insertion 12 of the calcanean tendon 13 and at right angle thereto, then a hollowed wick 14 is engaged on said spindle 11 and is used for drilling into the calcaneum 10 a hole 15 for receiving the screw 2.

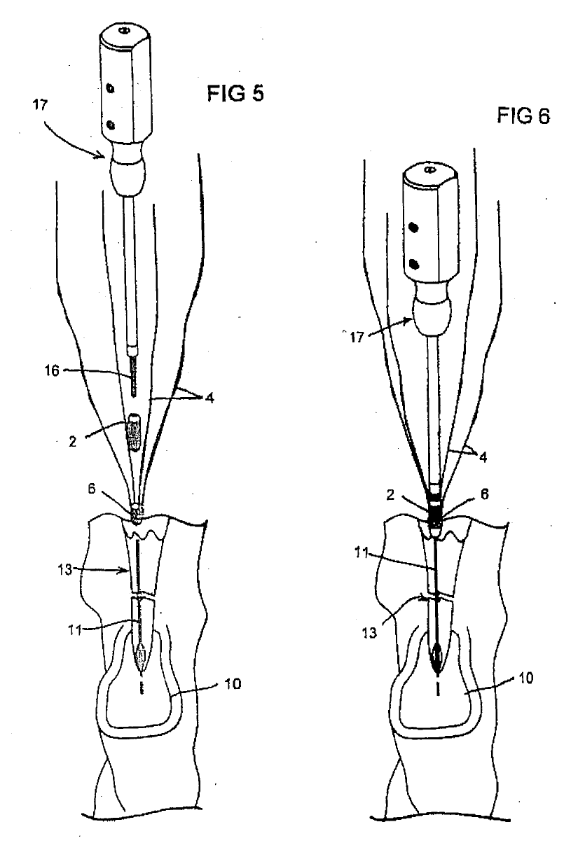

The screw 2 is engaged in the tubular part 6 so that the proximal third of the screw 2 lies in said tubular part 6, then the screw 2 is engaged on the prismatic end 16 of a hollow needle screwdriver 17, said end 16 being suited to the axial cavity of the screw 2, and the assembly is engaged on the spindle 11 (see FIGS. 5 and 6).

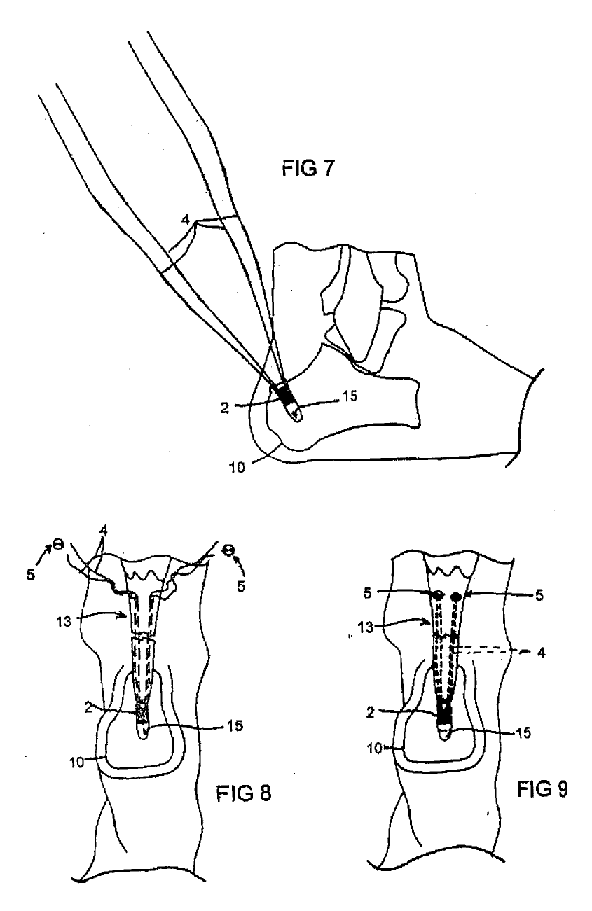

The screwdriver 17 is then used for tightening the screw 2 in the hole 15 until complete insertion in the hole of said screw 2 and of the tubular part 6, as shown on FIG. 7.

The spindle 11 is the removed and the cords are inserted into the fragments of the tendon 13, as shown on FIG. 8, then the buttons 5 are engaged, thanks to the holes which they include, on the respective pairs of cords 4.

The cords 4 are tensioned in order to coapt the fragments of the tendon 13, then ligatures of both cords 4 engaged through the same button 5 are realised in order to lock the button 5 with respect to these cords 4. The portions of cord 4 in excess are then cut off (see FIG. 9).

The invention thus provides a piece of equipment for repairing biological tissue, such as a tendon or a ligament, and in particular the calcanean tendon, which advantageously improves significantly the sequels of the intervention and the patient's comfort inasmuch as it enables more precocious physiotherapy whereby, in the case of a calcanean tendon, the patient may lay his foot on the ground straightaway, which provides for less perilous walking of the patient. Moreover, when the equipment in all or in part is bioresorbable, it becomes possible to leave the equipment remains in place in the organism until the resorption thereof, so that no new intervention is necessary to the ablation of the equipment and that the equipment remains efficient for a duration, of approximately three months, longer than the duration of implantation of a piece of equipment according to prior art, thereby enabling the patient to resume his activities earlier and more reliably, which proves more reassuring for the surgeon.

The invention is not limited to the embodiment described above by way of example, but encompasses all embodiments and variations covered by the claims. Thus the equipment may be used not only for repairing a calcanean tendon but also, in particular, for repairing the cap of the shoulder joint, for repairing cruciate ligaments of the knee and reinsertion of the biceps tendon. When treating the shoulder joint, the link may be in particular of non-bioresorbable material, whereas the screw and the stop buttons may be of bioresorbable or non-bioresorbable material.

1-7. (canceled)

8. Equipment for repairing biological tissue, such as a tendon or a ligament, and in particular a calcanean tendon, comprising:
a link whereof portions may be inserted through fragments of the biological tissue to be repaired, and
elements for stopping the link relative to such fragments of tissue;
equipment characterized in that:
one of the stop elements is formed of a bone screw which may be implanted in a bone surrounding the tissue to be repaired, in particular in the calcaneum in order to repair the calcanean tendon;
the link forms a tubular part in which one part of the screw can be tightly inserted along with at least two cords which can be inserted through the fragments of the biological tissue to be repaired, in particular the two fragments of a broken calcanean tendon, and
other stop elements comprise at least two stop buttons.

9. Equipment according to claim 8, characterized in that the screw and/or the link and/or the stop buttons are made of a bioresorbable material.

10. Equipment according to claim 8, characterized in that the screw is preferably a so-called “interference” screw, i.e. deprived of a head and controlled via a prismatic cavity emerging into the proximal end thereof.

11. Equipment according to claim 8, characterized in that the tubular part of the link is realised by knitting threads in the form of a sock, and the cords are realised by weaving said very threads.

12. Equipment according to claim 8, characterized in that the link includes four cords.

13. Equipment according to claim 8, characterized in that each cord exhibits a rigidified free end to easier insertion thereof through the eye of a needle.

14. Equipment according to claim 8, characterized in that each stop button comprises one or two through-holes, for the engagement thereof on a cord and the immobilisation thereof relative to the latter by ligature.
